(19) United States
(12) Patent Application Publication
(10) Pub. No.: US 2009/0053773 A1
(43) Pub. Date: Feb. 26, 2009

(54) Reaction Container and Dna Amplification Reaction Method

(75) Inventors: Rika Sato, Tokyo (JP); Kosuke Ueyama, Kuki-shi (JP)

Correspondence Address: STAAS & HALSEY LLP, SUITE 700, 1201 NEW YORK AVENUE, N.W., WASHINGTON, DC, 20005, US

(73) Assignees: TOPPAN PRINTING CO., LTD., TOKYO (JP); SHIMADZU CORPORATION, KYOTO (JP); RIKEN, WAKOU (JP)

(21) Appl. No.: 12/087,973

(22) PCT Filed: Jan. 20, 2006
(86) PCT No.: PCT/JP2006/300861

§ 371 Date: Jul. 18, 2008

(51) Int. Cl. C12P 19/34; C12M 1/00

(52) U.S. Cl. 435/91.2; 435/305.3; 435/91.1

(57) ABSTRACT

A linear recess is provided in a reverse side surface of a synthetic resin base plate 1, and a film 3 covers the linear recess. In this manner, a tunnel-shaped reaction chamber 12 surrounded by the film 3 and the base plate is configured. In addition, a protruding portion 31 is formed in a manner such that the film 3 protrudes into the reaction chamber 12 from the reverse side surface of the base plate 1. Since evaporation of a reagent and the like is suppressed and gaps which are caused by a difference in thermal expansion coefficients between the base plate 1 and the film 3 are formed only in side walls of the reaction chamber 12 and the protruding portion 31 of the film 3, reduction of the reagent and the like is prevented and a reaction product can be sufficiently obtained.

### TECHNICAL FIELD

The present invention relates to a disposable container suitable for biological reaction, for example, a disposable reaction container which can be applied when a sample including a minute amount of DNA is collected from a body, subjected to PCR amplification, and examined for a single nucleotide polymorphism thereof.

### BACKGROUND ART

A single nucleotide polymorphism (SNP) means that a single base is substituted with another base in a DNA sequence. Due to a difference of this single base, differences of individuals are generated, that is, differences in easiness of catching a disease and the like, and in effects and side-effects of administered drug. For this reason, SNP examination attracts attention to examine a physical constitution at a genetic level and direct a treatment or prevention policy in accordance with the physical constitution.

For example, DNA which is included in a sample such as blood collected from a body is used as DNA to be used in this examination. In order to collect a sample such as blood in a small amount and efficiently perform the examination, DNA in the collected sample is amplified and the amplified DNA is examined to detect SNP of DNA.

Various methods are known for amplifying a minute amount of DNA included in a sample, and a PCR method is known as a typical method thereof. In this method, three processes including: a denaturing process of double strand DNA in the sample (a single strand DNA is generated); an annealing process (oligonucleotide referred to as primer and a part of the single strand DNA are hybridized with each other); and an elongating process (nucleotide is elongated using the primer as a start point), are set as one cycle, and the cycle is repeated to amplify DNA in the sample. In theory, DNA can be amplified 2ⁿ-fold when n is the number of cycles. The denaturing process is performed at a temperature of 80° C. to 100° C., the annealing process is performed at a temperature of 50° C. to 60° C., and the elongating process is performed at a temperature of 60° C. to 80° C. Time for one cycle is at most about 10 minutes. However, several hours may be required to repeat the cycle to thereby amplify a required amount of DNA.

The amplified DNA is used in an examination process (typing process) of SNP included in the DNA. There are various typing methods, and a typical example thereof is an invader assay. In this method, two kinds of fluorescently unlabeled oligonucleotides (allele probe, invader probe), one kind of a fluorescently labeled oligonucleotides (FRET probe), and an endonuclease (cleavase) specific to a DNA structure are used. An allele probe is an oligonucleotide which has a sequence (flap) unrelated to a sequence of template DNA at the 5′ end thereof, and a complementary sequence specific to template DNA at the 3′ end. Herein, the 5′ end terminal of the complementary sequence is a SNP site. Meanwhile, an invader probe is designed so as to allow complementary binding from the SNP site to the 3′ side of the template DNA. Furthermore, a FRET probe is a fluorescent-labeled oligonucleotide which has a fluorescent label (Reporter) at a 5′ end thereof and Quencher is bonded on the upstream thereof. In addition, a 3′ end site from the Reporter is self-hybridized to constitute a double strand, and the 3′ end terminal from the double stranded part has a single stranded part which is a sequence complementary to the allele-probe flap. Cleavase is an enzyme which recognizes an area where nucleotides are triply overlapped, and cleaves a 3′ end of the triply overlapped nucleotides to releases the part.

In this invader assay, firstly, the 3′ end of the invader probe invades the SNP site when the template DNA, which is an examination subject, and the allele probe are hybridized with each other. Accordingly, at the SNP site, the template DNA, the allele probe, and the invader probe constitutes a tripartite structure. The cleavase recognizes the structure of the SNP site, and thereby the allele probe flap is cut and released. Next, the released flap originating from the allele probe is hybridized with the FRET probe. Due to this hybridization, at an intersection of the self-hybridized double strand with the released flap originating from the allele probe, a tripartite structure occurs. The cleavase recognizes the structure of the site again and cut the Reporter of the FRET probe to be released from the Quencher. Furthermore, excitation light is emitted such that the fluorescent-label of the cut and released Reporter emits fluorescence. In case where the base at the SNP site is not matched with the allele probe, the flap originating from the allele probe is not cut and released, and thus the fluorescence emitting ratio is considerably low. The SNP can be examined by detecting a difference in fluorescent light intensity. Generally, ultraviolet light or visible light is used as the excitation light.

Since the examination accuracy of such DNA examination technique is compromised by a contamination, there is proposed a method in which a disposable reaction container is used, the base plate thereof having a plurality of recesses as storage chambers, reaction chambers, and examination chambers. Required reagents and the like are stored in the storage chamber, a PCR amplification is performed in the reaction chamber using the reagents, and a typing reaction is performed in the examination chamber (Japanese Unexamined Patent Application, First Publication No. H05-317030). According to this method, since an examination of a single examination subject can be finished using the disposable reaction container, a contamination can be prevented, and an accurate examination can be performed.

Patent Document 1: Japanese Unexamined Patent Application, First Publication No. H05-317030

### DISCLOSURE OF THE INVENTION

However, as described above, the PCR amplification requires that the heat cycle in the range of 80° C. to 100° C. is repeated, and several hours are required to obtain a required amount of DNA. When heating is performed for such a long time, the DNA and the reagents may be evaporated, causing a reduction in volume, and the required amount of DNA cannot be obtained.

Accordingly, an object of the invention is to provide a disposable reaction container capable of obtaining a required amount of DNA by preventing evaporation of DNA and reagents and an amplification reaction method using the reaction container.

That is, the invention according to aspect 1 is a reaction container including a base plate and a film formed integrally with the base plate. The base plate has a linear recess on a reverse side surface thereof and the film covers the linear recess to configure a tunnel-shaped reaction chamber surrounded by the film and the base plate. The film enters the reaction chamber from the reverse side surface of the base plate to form a protruding portion.

The invention according to aspect 2 is the reaction container according to aspect 1, in which a height of the protruding portion is 0.1 μm to 10 μm.

The invention according to aspect 3 is the reaction container according to aspect 1 or 2, in which the film is formed integrally with the base plate by a curable adhesive.

The invention according to aspect 4 is the reaction container according to aspect 1 or 2, in which the film is formed integrally with the base plate by heat-sealing.

The invention according to aspect 5 is the reaction container according to any one of aspects 1 to 4, in which both ends of the tunnel-shaped reaction chamber have through holes penetrating the base plate.

The invention according to aspect 6 is the reaction container according to any one of aspects 1 to 5, in which the tunnel-shaped reaction chamber is a gene amplification reaction chamber.

The invention according to aspect 7 is a gene amplification reaction method including injecting a sample including a gene, a gene amplification reagent, and a nonvolatile liquid having a light specific gravity into the reaction chamber of the reaction container according to aspect 5; and performing amplification reaction of the gene.

The invention according to aspect 8 is the gene amplification reaction method according to aspect 7, in which the sample is a biological sample.

The invention according to aspect 9 is the gene amplification reaction method according to aspect 7 or 8, in which the gene amplification reagent is a PCR reaction reagent.

The invention according to aspect 10 is the gene amplification reaction method according to any one of aspects 7 to 9, in which the nonvolatile liquid is mineral oil, vegetable oil, or silicon oil.

### BRIEF DESCRIPTION OF THE DRAWINGS

FIG. 1A is an exploded perspective view showing an example of a reaction container according to the invention, and FIG. 1B is a reverse-side perspective view of a base plate.

FIG. 2 is a main portion cross-sectional view showing a storage chamber.

FIG. 3 is a main portion cross-sectional view showing a reaction chamber.

FIG. 4 is a main portion cross-sectional view showing an examination chamber.

FIGS. 5A to 5C are plan views for illustrating a shape of peeling guiding projections.

FIGS. 6A to 6B are plan views for illustrating a shape of the peeling guiding projections.

FIG. 7 is a perspective view showing another example of the base plate according to the invention, and FIG. 7B is a reverse-side perspective view thereof.

### BEST MODE FOR CARRYING OUT THE INVENTION

A reaction container according to the invention is configured including a base plate and a film as essential parts. The base plate has a linear recess provided on a reverse side surface thereof and the film covers the linear recess to configure a tunnel-shaped reaction chamber surrounded by the film and the base plate. The tunnel-shaped reaction chamber can be used in, for example, gene amplification reaction such as DNA or RNA. The film enters the reaction chamber from the reverse side surface of the base plate to form a protruding portion. The protruding portion may be small, and the height thereof may be, for example, 0.1 μm or more. Moreover, it is preferable that the height is 10 μm or less. PCR amplification for DNA is a typical example of the amplification reaction.

In addition to the tunnel-shaped reaction chamber, separate examination chambers may be provided on the base plate. Furthermore, storage chambers may be provided on the base plate to store reagents to be used for the chemical reaction in the reaction chamber and the examination chamber. The examination chamber and the storage chamber may be covered by the film or exposed without being covered.

For example, a plurality of the storage chambers and the examination chambers can be provided on the base plate to store amplification reagents to be used in amplification reaction in some storage chambers, and to store diluents to be applied to the amplification reaction in other storage chambers. In this case, the reaction chamber serves as a PCR amplification chamber for PCR amplification. The examination subject obtained by amplifying specimen DNA in the PCR amplification chamber is dispensed into the plurality of the examination chambers, and thus different typing reactions can be performed in each examination chamber. Furthermore, in this case, it is preferable that the plurality of the storage chambers and the PCR amplification chambers are sealed by a cover material. In addition, typing reagents can be stored in some of the storage chambers or stored in the plurality of the examination chambers in advance. By storing the diluents or the PCR reagents to be applied in the PCR amplification in the storage chambers and storing the typing reagents in the examination chambers in advance, the entire process of SNP examination can be performed on the reaction container. Accordingly, human errors such as mistakes in the PCR amplification process and the typing process or use of improper reagents can be prevented. Consequently, the SNP examination can be accurately performed.

The base plate can be manufactured by injection molding of synthetic resin. The synthetic resin may be any synthetic resin enduring reaction conditions such as heat upon reaction and not disturbing accurate reaction. In case where the reaction container is used for typing reaction of DNA, it is preferable to use synthetic resin having a high transmission factor of excitation light (ultraviolet light or visible light) and fluorescent light (visible light). For example, as for FAM which is known as a fluorescently-labeled substance, the wavelength of the excitation light is 494 nm and the wavelength of the fluorescent light is 518 nm. As for RED, the wavelength of the excitation light is 579 nm and the wavelength of the fluorescent light is 595 nm. Preferably, the base plate has a transmission factor of excitation light and fluorescent light of 70% or more, and more preferably 85% or more.

As such synthetic resin, for example, polyolefin resin such as polyethylene or polypropylene can be preferably used. Acrylic synthetic resin such as polymethylacrylate or polymethylmethacrylate also can be used. In addition, polyester synthetic resin such as polycarbonate or polyethylene terephthalate, polyvinyl chloride resin, or polystyrene resin also may be used.

A thickness of the base plate is preferably 2 mm or less for the same reason. The transmission factor of excitation light and fluorescent light is reduced when the thickness exceeds 2 mm. It is preferable that the thickness is 1 mm or less. It is preferable that the thickness is at least 0.3 mm to prevent heat deformation.

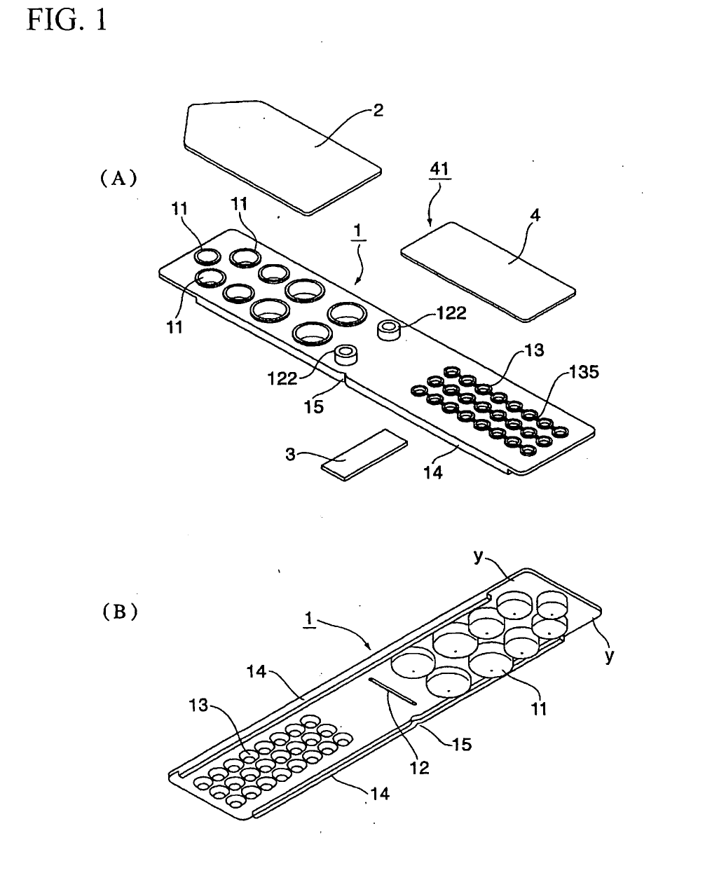

FIG. 1A shows an example of the reaction container according to the invention. The reaction container is configured by a base plate 1, a film cover material 2 for storage chambers, a reaction chamber forming film 3, and an examination chamber protecting film 4. The base plate 1 has a transversely long rectangular shape. The long sides of the base plate 1 are 5 cm to 15 cm and the short sides thereof are 1 cm to 5 cm. Nine storage chambers 11, a single PCR amplification reaction chamber 12, and twenty-four examination chambers 13 which are arranged in three columns by eight lines are provided sequentially along the longitudinal direction of the base plate 1. A cutout portion 15 is provided at one of the sides in the longitudinal direction to prevent errors in taking the direction of the base plate 1. Ribs 14 are provided along both the sides on the reverse side to prevent deformation such as warpage along the longitudinal direction. FIG. 1B is a perspective view of the base plate 1, viewed from the reverse side.

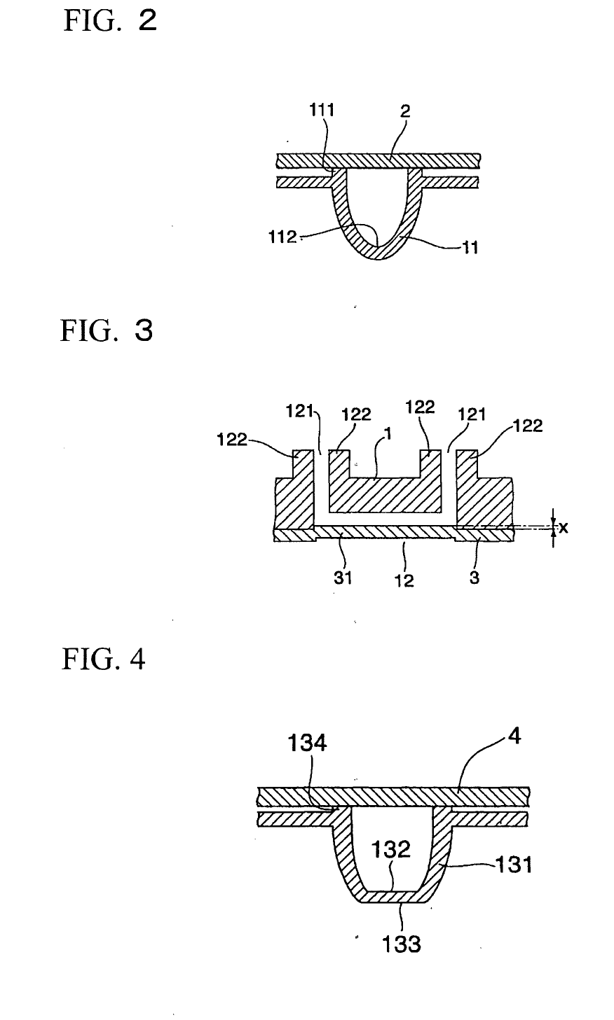

All of the nine storage chambers 11 store chemicals to be applied to the PCR amplification reaction. That is, some of the storage chambers 11 store PCR reagents including polymerase, and some other storage chambers 11 store diluents. Since the PCR reagents and the diluents are liquid, the storage chambers 11 are liquidtightly sealed with the film cover material 2 (see FIG. 2). The storage chambers 11 are provided in a shape of a recess on the base plate 1 and an inner volume thereof is larger than an inner volume of the examination chambers to be described later. For example, a diameter of an opening of each storage chamber 11 is 5 mm to 10 mm and a depth thereof is 5 mm or less. Linear projections 111 protruding from the base plate 1 are provided around the openings. Heights of the projections 111 are the same to each other and the film cover material 2 is adhered to each of the projections 111 across all of the projections 111.

All of the storage chambers 11 have an inner wall formed in a taper shape such that a cross-section area is gradually reduced toward the bottom 112. Even in case where an amount of the reagents and the like stored in the storage chambers 11 is small, the reagents and the like are collected at a predetermined portion, that is, at a center of the bottom 112. In case where the reagents and the like are always collected at the predetermined position as described above, the reagents and the like can be easily and securely removed by using an injector-shaped syringe or a pipette. Each storage chamber 11 may be configured such that an upper portion of an inner surface includes a uniform cross-section area and the bottom 112 is tapered. In this case, the reagents and the like also can be collected at the predetermined portion or easily and securely removed.

In addition, it is preferable that at least the bottom 112 of each storage chamber 11 has superior affinity to the reagents and the like to be stored. In case where the bottom has low affinity, the reagents and the like are separated in small spheres and dispersed to respective positions by surface tension if amounts of the reagents and the like are small. In case where the reagents and the like have a hydrophilic property, the bottom 112 of each storage chamber can be subjected to a hydrophilic treatment to increase the affinity for the reagents and the like. Examples of the hydrophilic treatment include an atmospheric plasma treatment, a corona discharge treatment, and a surface treatment with oxidizing chemicals such as an ozone gas.

Moreover, the surface energy of the bottom 112 of each storage chamber is increased by providing with fine unevenness. As a result, the affinity to the hydrophilic reagents and the like is improved and the stored reagents and the like can be collected at the predetermined position. The fine unevenness can be formed by, for example, performing a sandblast treatment on the bottom surface. Furthermore, the fine unevenness of the bottom 112 can be formed by irradiating with laser light. In addition, the bottom 112 of each storage chamber can be provided with fine unevenness by manufacturing the base plate 1 by injection molding with the use of a mold of which the area corresponding to the bottom 12 is treated by a sandblast treatment. A ten-point average roughness of the fine unevenness is preferably 1.0 μm or more, and more preferably 1.5 μm or more. In addition, the ten-point average roughness of the unevenness is preferably 100 μm or less, and more preferably 30 μm or less.

When a hydrophilic reagent of 58.0 μl was stored in the storage chamber having a theoretical capacity of 96.6 μl and the reagent was removed by jabbing an injector-type tip or a pipette, the reagent remaining on the bottom 112 was 17 μl to 38 μl (collection rate of 70% to 34%) in case where the bottom 112 of each storage chamber was flat (ten-point average roughness of about 0.2 μm). On the other hand, a base plate 1 provided with an unevenness having a ten-point average roughness of 2 μm on the bottom 112 of each storage chamber was used as a control experiment. This base plate 1 is prepared by performing an injection molding with a mold, which was subjected to a sandblast treatment by using a sand count A220 (2 μm) under the following conditions: a pressure of 4 kg/mm; and an emission distance of 15 cm. In this control experiment, the reagent remaining on the bottom was 9 to 17 μl (collection rate of 84% to 70%).

As described above, it is convenient when the storage chambers 11 store the reagents and the like to be used in advance (however, it is preferable that at least one storage chamber is empty. This empty storage chamber is used as a portion for storing a sample such as blood collected from a human body). Since the PCR reagents and the diluents are liquid, it is preferable that some of the storage chambers 11 store the liquid reagents and the like, and are liquidtightly sealed with the cover material 2. A synthetic resin injection-molded product can be used as the cover material 2. However, as in this example, it is preferable that a film cover material is used to be adhered to projections 111 around the openings of the storage chambers 11. The storage chambers 11 can be covered with a plurality of individual film cover materials. However, since the projections 111 are linearly configured and have the uniform height, it is preferable that one large film cover material 2 covers and is adhered to all of the projections 111 of the storage chambers 11. Furthermore, the reagents and the like in the storage chambers can be removed by, for example, using an injector-shaped syringe or the like, jabbing through the film cover material 2. In case where a sample such as blood collected from a body is stored, the injector-shaped syringe or the like can be jabbed to store the sample in the storage chambers. Consequently, the film cover material 2 is not required to be removable from the base plate. However, the film cover material 2 can also be adhered in a removable manner to the projections 111. In this case, a part or all of the film cover material 2 can be removed such that the storage chambers 11 can be used in an exposed state.

A synthetic resin film can be used as the film cover material 2 for the storage chambers. Examples of such synthetic resin film include a polyolefin film such as polyethylene, polypropylene, and polymethylpentene, an acrylic synthetic resin film such as polymethylacrylate and polymethylmethacrylate, a polystyrene film, a polyacetal film, a polyamide film, a polyacrylonitrile film, a polycarbonate film, a polycycloolefin film, a silicon resin film, a fluoroplastic resin film, and the like. In addition, a metal foil or a laminated film in which a synthetic resin film is laminated on a metal foil can be used as the film cover material 3. The film cover material 2 may be transparent or opaque.

The film cover material 2 for the storage chambers can be adhered to the projections 111 by using, for example, a heat-resistant adhesive. The adhesive includes, for example, a curable adhesive. Examples of such curable adhesive include an epoxy adhesive, an urethane adhesive, and the like. Also, a light curing adhesive including an acrlic monomer and a photoinitiator can be used. In addition, adhesion can be performed by heat-sealing.

Next, the PCR amplification reaction chamber 12 is a portion in which the PCR amplification is performed. As shown in a main portion cross-sectional view of FIG. 3, the PCR amplification reaction chamber 12 is configured in a tunnel shape. That is, the reverse side surface of the base plate 1 has a linear recess and the reaction chamber forming film 3 is adhered to the circumstance of the linear recess so as to cover the linear recess. As a result, a tunnel-shaped portion surrounded by the reaction chamber forming film 3 and the base plate 1 is formed and the tunnel-shaped portion serves as the PCR amplification reaction chamber 12. As described above, the PCR amplification may be performed for more than an hour at a high temperature. However, the evaporation of a reaction liquid can be prevented, in a manner such that the reaction is performed in the tunnel-shaped PCR amplification reaction chamber 12 having a high hermetic performance and a nonvolatile liquid having a lighter specific gravity than the sample and the reagent is injected to cover a surface thereof. As the nonvolatile liquid, mineral oil, vegetable oil, or silicon oil can be used.

Furthermore, through holes 121 which penetrate the base plate 1 are provided at both ends of the tunnel-shaped PCR amplification reaction chamber 12. Furthermore, tubular protruding portions 122 each of which has a central hole communicating with the through hole 121 are provided on the surface of the base plate 1. By the communication of the central holes of the tubular protruding portions 122 with the through holes 121, the PCR reagents, the diluents, and the nonvolatile liquid can be injected into the tunnel-shaped PCR amplification reaction chamber 12 and the examination subject obtained by the amplification can be removed from the tunnel-shaped PCR amplification reaction chamber 12. A protecting film (not shown) can be adhered on the tubular protruding portions 122 to prevent the central holes from being contaminated.

The height of the tunnel-shaped PCR amplification reaction chamber 12, that is, the depth of the linear recess is preferably in the range of 0.1 mm to 5.0 mm. When the depth is shallower than this range, it is difficult that a required amount of the examination subject for being dispensed into the examination chambers is generated by the PCR amplification. When the depth is deeper than this range, heat for the PCR amplification is not sufficiently transmitted and there is a possibility that the required amplification reaction is not performed.

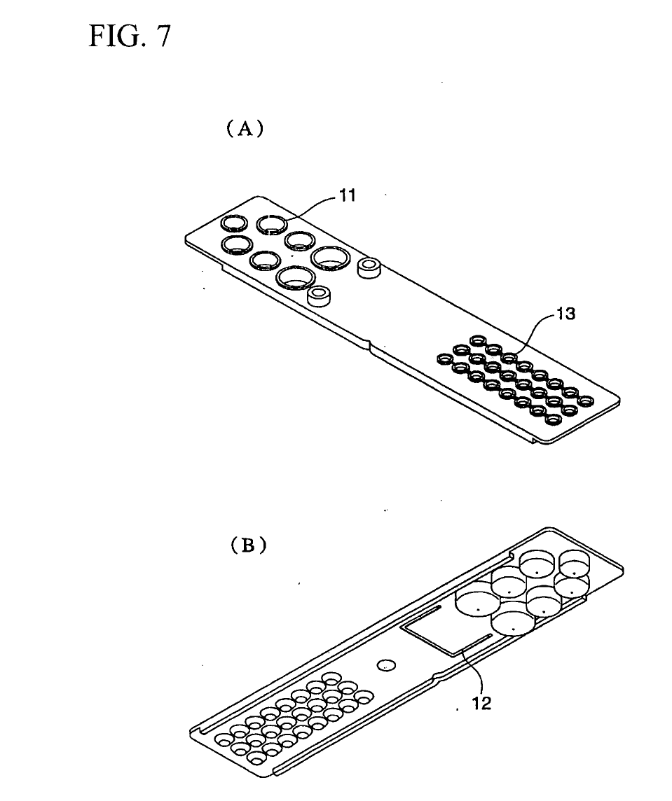

The tunnel-shaped PCR amplification reaction chamber 12 may have a shape such that the through holes 121 are connected to each other by a straight line. However, it is preferable that the tunnel-shaped PCR amplification reaction chamber 12 has a shape of a curved line between the through holes 121 to suppress the evaporation of the reaction liquid. For example, the PCR amplification reaction chamber 12 may have an arc shape, a zigzag shape, an U-shape, or a combined shape thereof. FIG. 7A is a perspective view of a base plate in which a tunnel-shaped PCR amplification reaction chamber 12 having a U-shape is provided and FIG. 7B is a perspective view of a reverse side thereof.

In general, it is preferable that the reaction chamber forming film 3 which configures the PCR amplification reaction chamber 12 by covering the linear recess slightly protrudes into the PCR amplification reaction chamber 12 from the reverse side surface of the base plate 1 at the position in the linear recess to configure a protruding portion 31. In general, thermal expansion coefficients of the base plate 1 and the film 3 are different from each other. Accordingly, a heat cycle of three steps for the PCR amplification (DNA denaturing step, annealing step, elongating step) may cause gaps to form between the base plate 1 and the film 3. Even in case where such gaps are caused, the gaps are caused only at the side walls of the PCR amplification reaction chamber 12 and a protruding portion 31 of the film 3, but not caused between the base plate 1 and the film 3, since the film 3 is protruding from the reverse side surface of the base plate 1 to the PCR amplification reaction chamber 12. A height x of the protruding portion 31 may be 0.1 μm to 10 μm.

A film capable of being elongated by pressing can be preferably used as the reaction chamber forming film 3. For example, a thermoplastic synthetic resin film can be used. The film 4 may be transparent or opaque. A laminated film which has a metal foil and a synthetic resin film laminated on the metal foil can also be used. As such thermoplastic metal foil, for example, an aluminum foil can be preferably used. When the laminated film including the metal foil is used, the PCR amplification can be efficiently performed, since the laminated film has a high steam barrier property and an excellent heat transfer property upon reaction.

The reaction chamber forming film 3 can be adhered to the base plate 1 by using, for example, a heat-resistant adhesive. The adhesive includes, for example, a heat-curable adhesive. Examples of such heat-curable adhesive include an epoxy adhesive, an urethane adhesive, and the like. Also, a light curing adhesive including an acrlic monomer and a photoinitiator can be used. In addition, adhesion can be performed in a manner such that one surface of the reaction chamber forming film 3 is entirely covered with a curable adhesive; the adhesive surface is placed on the base plate 1; the reaction chamber forming film 3 is slightly elongated and protruded into the PCR amplification reaction chamber 12, by pressing it against a pressing die having a protruding portion in the vicinity of the center of the bottom of the tunnel-shaped PCR amplification reaction chamber 12; and by heating or ultraviolet irradiation in order to cure in this state. In this case, the cured adhesive is exposed on the bottom surface of the PCR amplification reaction chamber 12. However, since the adhesive is already cured, the PCR amplification can be accurately performed without being inhibited by the adhesive.

Furthermore, the reaction chamber forming film 3 can be adhered to the base plate 1 by heat-sealing. That is, the film 3 can be adhered to the base plate 1, in a manner such that the thermoplastic resin film is: placed on the base plate 1 facing the base plate 1; then pressed with a pressing die having a protruding in the vicinity of the center of the bottom of the tunnel-shaped PCR amplification reaction chamber 12; and then subjected to heating. In this case, the film 3 is exposed on the bottom surface of the PCR amplification reaction chamber 12 and the PCR amplification can be accurately performed without being disturbed.

Next, the examination chambers 13 will be described. The twenty-four examination chambers 13, which are arranged in three columns by eight lines as shown in the figure, are a portion for performing the typing reaction. The number of the examination chambers 13 is not limited to twenty four. However, since the number of SNP to be examined varies according to the examination subject and the reagent to be used varies according to each SNP, it is preferable that the number of the provided examination chambers 13 is the same as the number of the various reagents. By storing different kinds of typing reagents in the plurality of the examination chambers 13 in advance, the typing reaction of each examination chamber 13 can be specified and thereby examination errors can be prevented.

As shown in a main portion cross-sectional view of FIG. 4, the examination chambers are provided in a shape of a recess on the base plate 1. It is preferable that the examination chambers 13 for performing the typing reaction include recesses having smaller internal capacity than the storage chambers 11. For example, a diameter and a depth of an opening of each examination chamber may be 5 mm or less, and preferably 0.01 mm to 5 mm. This is because the typing reaction uses a minute amount of reaction product in which the DNA is amplified in the PCR amplification chamber 13 as the examination subject, and because an accurate reaction using such an minute amount of examination subject is required. It is also required that the excitation light emitted from the reverse side surface of the base plate 1 is accurately condensed on the examination subject, fluorescent light is precisely generated by the condensed excitation light, and the fluorescent light is precisely detected by a detection device. If the minute amount of the examination subject is reacted in a large reaction chamber, there is a danger that the fluorescent light generated by the excitation light is too weak to be detected.

Furthermore, it is preferable that, by forming the side wall 131 of each examination chamber 13 into a tapered shape, air bubbles are prevented from being formed upon dispensing of the examination subject in which the DNA is amplified in the PCR amplification chamber 13, and the examination subject is securely stored at the bottom of each examination chamber 13. And it is preferable, by forming a bottom surface 132 of each examination chamber 13 into a flat shape, to prevent refraction and deflection of the excitation light emitted from the reverse side of the base plate 1. For the same reason, it is preferable that a reverse side surface 133 of the base plate 1 facing the bottom surface is also a flat surface parallel to the bottom surface 132.

In order to ensure an transmission factor of the excitation light and the fluorescent light of 70% or more, a distance between the bottom surface 132 and the reverse side surface 133 of the base plate 1 facing the bottom surface 132 (the thickness of base plate 1) is preferably 2 mm or less, and more preferably 1 mm or less.

To prevent the air bubbles from being formed upon dispensing of the examination subject, a preferable angle between the bottom surface 132 and the side wall 131 is in the range of 100 degrees to 140 degrees. The typing reagents can be stored and provided in a solid state so as to be in contact with the bottom surface 132 of each examination chamber 13.

To seal the examination chambers 13 and prevent contamination thereof, it is required that linear projections 134 are provided around the openings of the recesses configuring the examination chambers 13, and that the protecting film 4 is removably adhered to the projections 134 to integrate the film with the base plate 1. The protecting film 4 is removed before the examination chambers 13 are used.

Since the protecting film 4 is adhered to all of the twenty-four examination chambers 13, the film is required to have a size capable of covering all of the twenty-four examination chambers 13. All of the twenty-four examination chambers 13 can be opened at one time by peeling off the film from an end portion thereof.

The reaction container is required to have peeling-guiding projections along the designed peeling direction of the protecting film 4. As described above, the peeling-guiding projections can be provided independently of the linear projections 134 around the openings. However, in the example shown in the figure, the peeling-guiding projections include the projections 134 disposed around the recesses as a part thereof. The peeling-guiding projections are configured including the projections 134 disposed around the recesses, and connecting projections 135 connecting the projections to each other. The protecting film 4 is adhered to the peeling-guiding projections to guide the peeling of the protecting film 4. That is, by adhering the protecting film 4 to all of the peeling-guiding with a uniform adhesive property, the peeling can be performed with a constant force from the start to the end of the peeling and all of the twenty-four examination chambers 13 can be thereby opened at one time.

For such reason, it is preferable that all of the projections 134 and the connecting projections 135, configuring the peeling-guiding projections, and disposed around the recesses, are linear. In this case, the protecting film 4 and the linear projections can be adhered to each other with an uniform pressure exerted on the protecting film 4. In addition, the adhesive property between the film and the linear projections which are adhered to each other under the uniform pressure is also uniform and the peeling can be performed with a constant force from the start to the end of the peeling.

Furthermore, it is preferable that the projections 134 configuring the peeling-guiding projections and disposed around the recesses, and the connecting projections 135 are substantially equal in width. In this case, the peeling can be performed with a constant force and without breaking from the start to the end of the peeling. Preferably, when a width of the narrowest portion is 100%, a width of the largest portion does not exceed 200%.

All of the width of the projections 134 disposed around the recesses and the width of the connecting projections 135 are preferably 0.1 mm to 3 mm. The protecting film 4 can be naturally peeled off without being broken on the way when the widths of the projections 134 and the connecting projections 135 are 0.1 mm to 3 mm.

When the peeling-guiding projections are configured independently of the projections 134 disposed around the recesses, it is preferable that all of the peeling-guiding projections and the projections 134 disposed around the recesses are linear. Moreover, it is preferable that top portions of the peeling-guiding projections (that is, portions adhering to the protecting film 4) are substantially equal in width. Furthermore, the widths of the peeling-guiding projections and the projections 134 disposed around the recesses are preferably 0.1 mm to 3 mm.

The cross-sectional shape of the peeling-guiding projections can be a rectangular shape, a trapezoidal shape, or a sector shape with an arc or elliptical outer line. The cross-section shape of the projections 134 configuring the peeling-guiding projections and disposed around the recesses and the cross-section shape of the connecting projections 135 may be different from each other.

The area of the peeling-guiding projections is preferably 1% to 80% of the area of the protecting film 4. When the area of the peeling-guiding projections is larger than this range, the peeling becomes difficult. When the area of the peeling-guiding projections is smaller than this range, the adhesive strength becomes weak. In case where the peeling-guiding projections are configured independently of the projections 134 disposed around the recesses, the total area of these projections is in the range of 1% to 80%.

Next, the peeling-guiding projections are not required to be configured by one linear projection as a whole. For example, the peeling-guiding projections may be configured by a plurality of linear projections independent of each other. In any case, it is preferable that the peeling-guiding projections are continuously arranged along the designed peeling direction from the start position to the end position of the peeling. In this case, by performing the peeling toward the peeling direction, the entire protecting film 5 can be peeled off without being broken on the way and the plurality of the recesses can be thereby exposed. When the designed peeling direction is a longitudinal direction of the reaction chamber, a direction perpendicular to the designed peeling direction is the short side direction, and thus the force upon peeling does not disperse in the direction perpendicular to the peeling direction. Accordingly, the film can be peeled off over the whole of the short side direction without being torn apart along the designed peeling direction.

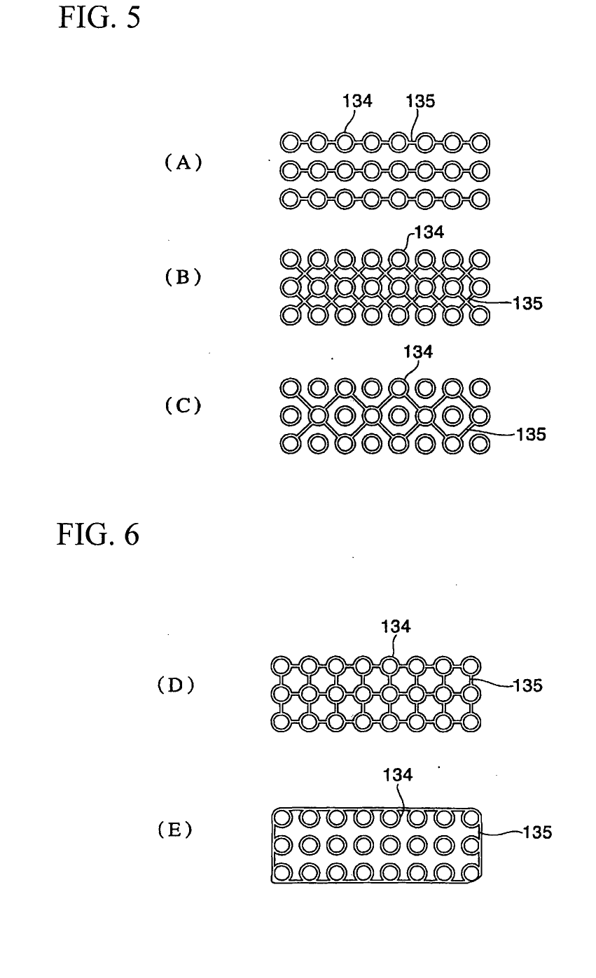

In the example of FIG. 1, twenty-four examination chambers 13 in total of three columns by eight lines are arranged in the longitudinal direction of the reaction chamber and the longitudinal direction (transverse direction in the figure) is set as the designed peeling direction. Accordingly, the connecting projections 135 are provided so as to connect the projections 134 disposed around the recesses included in the columns to each other, so as to form three independent linear projections from each other, the combination thereof as a whole serving as the guiding portions. FIG. 5A is a plan view for illustrating a shape of the connecting projections 135 of FIG. 1.

FIG. 5B is a plan view for illustrating another shape of the connecting projections 135. The twenty-four examination chambers 13 in total are continuously formed as a whole over the whole of the designed peeling direction such that the projections 134 of the examination chambers 13 obliquely adjacent to each other are connected to each other by the X-shaped connecting projections 135. In the example of FIG. 5C, the examination chambers 13 of each column are alternately connected such that, the second examination chamber 13 in the second column is connected to: the first examination chambers 13 in the first and the third columns; and the third examination chambers 13 of the first and third columns. The projections 134 disposed around the recesses and the connecting projections 135 are continuously formed as a whole over the whole of the designed peeling direction while forming zigzag lines. Furthermore, the examination chambers 13 adjacent to each other can be connected by the connecting projections 135 and thereby can be formed into a matrix shape in which all of the examination chambers 13 are connected to each other as a whole (see FIG. 6D). Moreover, among the twenty-four examination chambers 13, eighteen examination chambers 13 disposed on the edges can be connected and thereby continuously formed as a whole over the whole of the designed peeling direction (see FIG. 6E).

Next, it is preferable that the protecting film 4 has a non-adhered portion serving as a peeling-starting portion 41 on a part thereof. It is preferable that the peeling-starting portion is disposed at an end portion in the longitudinal direction of the protecting film 4. In the example of FIG. 1, since the transverse direction of the protecting film 5 is longitudinal, it is preferable that the peeling-staring portion is provided at the left end portion in the figure (that is, at the end of the storage chambers 11 side). In this case, the peeling starts from this starting position without an error in selecting a peeling-starting position, and the entire protecting film 4 is thereby precisely peeled off. Consequently, all of the recesses 13 can be exposed.

In addition, it is preferable that the peeling-starting portion 41 is disposed on a line which connects the portions at which the protecting film 4 is adhered. In this case, since the protecting film 4 is adhered and fixed at both sides of the peeling-starting portion, the peeling-starting portion 41 therebetween are also secured without becoming loose by the tension between the adhered portions. Consequently, the peeling can be easily performed by securely gripping the film upon starting of the peeling.

Next, it is preferable that all of the top portions of the peeling-guiding projections (that is, portions adhering to the protecting film 4) configure a plane or a smooth curved plane without a step. In this case, in adhering of the protecting film 4, the protecting film 4 can be uniformly and securely adhered to all of the peeling-guiding projections. Moreover, it is preferable that the projections 134 disposed around the recesses and the peeling-guiding projections are substantially equal in height, regardless of whether or not a part of the peeling-guiding projections are configured by the projections 134 disposed around the recesses. Even when there is a portion having a different height, the height of the highest portion is 150% or less of the height of the lowest portion.

Furthermore, it is preferable that the height of the peeling-guiding projections is 0.05 mm or more. When the peeling-guiding projections are configured independently of the projections 134 disposed around the recesses, the height of the peeling-guiding projections and the height of the projections 134 disposed around the recesses are 0.05 mm or more. In this case, in adhering of the protecting film 4, the film is adhered to the peeling-guiding projections and the projections 134 disposed around the recesses without being adhered to a portion other than the projections. Accordingly, the peeling can be performed with a constant force and without breaking from the start to the end of the peeling. Preferably, the height is 0.05 mm to 2 mm.

Next, examples of the protecting film 4 can include a polyolefin film such as polyethylene, polypropylene, and polymethylpentene, an acrylic synthetic resin film such as polymethylacrylate and polymethylmethacrylate, a polystyrene film, a polyacetal film, a polyamide film, a polyacrylonitrile film, a polycarbonate film, a polycycloolefin film, a silicon resin film, a fluoroplastic resin film, and the like. In addition, a metal foil or a laminated film in which a synthetic resin film is laminated on a metal foil can be used as the protecting film 5. The protecting film 4 may be transparent or opaque.

The protecting film 4 can be removably adhered to the recesses by, for example, heat-sealing. To adjust an adhesive strength thereof to thereby easily perform the peeling, it is preferable that different kinds of resin materials are applied to a heat-sealing surface of the protecting film 4 and the base plate 1, respectively. For example, when the base plate 1 is made of polypropylene, a polyethylene film or a laminated film of which a heat-sealing surface is made of polyethylene can be used as the protecting film 4.

However, since the base plate 1 has a long rectangular shape with the long side of 5 cm to 15 cm and the short side of 1 cm to 5 cm as described above, deformation such as warpage may occur by heat during molding, heat during the heat-sealing of the reaction chamber forming film 3 and the examination chamber protecting film 4, or heat during the PCR amplification or the typing reaction. When deformation such as warpage occurs in the base plate 1 of the portion where the examination chambers 13 exist, the bottom surfaces 132 of the examination chambers 13 and the reverse side surface 133 of the base plate 1 facing the bottom surfaces are tilted and excitation light emitted through the reverse side surfaces of the examination chambers 13 is refracted and deflected due to the tilt. Furthermore, a distance from an excitation light source is changed and a position of the examination subject and a condensing position are thereby displaced, and the fluorescent light generated by the excitation light is refracted and deflected. As a result, it is difficult to accurately perform the examination. Accordingly, it is preferable that the bottom surfaces 132 of the plurality of the examination chambers 13 are disposed on the same plane. Even when the surfaces are tilted, normal lines perpendicular to the bottom surfaces 132 of the examination chambers 13 disposed at both the ends among the plurality of examination chambers 13 makes an angle of 4 degrees or less, and preferably 1 degree or less. A difference in height of the bottom surface 132 of the examination chambers 13 disposed at both the ends is 4.0 mm or less, and preferably 1.0 mm or less.

The deformation-preventing ribs 14 prevent such deformation to hold the bottom surfaces of all of the examination chambers 13 on the same plane, and are provided in the both sides along the longitudinal direction of the portion in which the examination chambers 13 exist. The deformation-preventing ribs 14 may be provided over the whole length of the sides in the longitudinal direction of the reaction chamber beyond the portion in which the examination chambers 13 exist. However, it is preferable that sides y in the longitudinal direction of the end portion on the side of the storage chambers 11, as well as the top and reverse side surfaces of the base plate 1, are flat, in order to be used upon gripping the reaction container.

In the example of FIG. 1, the deformation-preventing ribs 14 are provided in the reverse side surface of the base plate 1. However, the ribs also can be provided in the top surface of the base plate 1 or in both the top surface and the reverse side surface. In addition, the deformation-preventing ribs 14 can be provided in a shape of a linear projection protruding in the top or reverse side surface of the base plate 1. When a thickness of the base plate 1 is 0.3 mm to 2 mm, a height of the deformation-preventing ribs 14 is preferably 0.1 mm to 5 mm and a width thereof is preferably 0.5 mm to 5 mm.

Herein, a polypropylene base plate having long sides of 10.0 cm, short sides of 2.5 cm, and a thickness of 0.5 mm was injection molded and an amount of warpage thereof was measured. The measurement was performed as follows. That is, two flat supporting stands were provided and fixed such that upper surfaces thereof are level. The surface was set as a reference surface. Then, both ends of the base plate were disposed on the two supporting stands and a distance between the center of the reverse side surface of the base plate 1 and the reference surface was measured as an amount of warpage. In addition, an angle made by the normal lines perpendicular to the bottom surfaces of the examination chambers 13 disposed at both the ends was measured.

In case of a base plate without the deformation-preventing ribs 14, the amount of warpage just after the injection molding was 0.9 mm and the crossing angle was about 0 degree 55 minutes. Next, the reaction chamber forming film 3 and the examination chamber protecting film 4 were subjected to heat-sealing at 190° C. for 5 seconds. The amount of warpage was 4.2 mm and the crossing angle was about 4 degrees 20 minutes.

Next, a polypropylene base plate having the deformation-preventing ribs 14 in both long sides thereof was injection molded with the same dimensions. The deformation-preventing ribs 14 were formed on the top side of the base plate. A height of the ribs was about 8.0 cm from the end portion on the side of the examination chambers 13, and both the sides y of the end portion of the storage chambers 11 were flat without having the deformation-preventing ribs 14. Furthermore, the deformation-preventing ribs 14 had a height of 1.0 mm and a width of 1.0 mm. The amount of warpage just after the injection molding was 0.9 mm and the crossing angle was about 0 degree 55 minutes. When the reaction chamber forming film 3 and the examination chamber protecting film 4 were subjected to heat-sealing under the same conditions, the amount of warpage was 3.2 mm and the crossing angle was about 3 degrees 20 minutes.

Furthermore, a polypropylene base plate having the deformation-preventing ribs 14 in both long sides thereof was injection molded with the same dimensions. The deformation-preventing ribs 14 were formed on the top side of the base plate. The height of the ribs was about 8.0 cm from the end portion on the side of the examination chambers 13 and the both sides y of the end portion of the storage chambers 11 were flat without having the deformation-preventing ribs 14. Furthermore, the deformation-preventing ribs 14 had a height of 1.0 mm and a width of 1.5 mm. The amount of warpage just after the injection molding was 0.4 mm and the crossing angle was about 0 degree 15 minutes. When the reaction chamber forming film 3 and the examination chamber protecting film 4 were subjected to heat-sealing under the same conditions, the amount of warpage was 0.75 mm and the crossing angle was about 0 degree 45 minutes.

As described above, as for the invention according to aspect 1, the linear recess in the reverse side surface of the base plate is covered such that the tunnel-shaped portion surrounded by the film and the base plate serves as the reaction chamber. Consequently, a sealing property increases, evaporation of the reaction liquid and the like is suppressed, and reduction of the reaction liquid and the like can be suppressed even when heating reaction is performed for long time.

Furthermore, since the film enters the reaction chamber from the reverse side surface of the base plate, gaps caused by a difference in thermal expansion coefficient between the base plate and the film are caused only in the side wall of the reaction chamber and the protruding portion of the film. Consequently, a loss of the reagents and the like by entering into the gaps can be reduced.

In addition, for the above reason, the reaction product can be sufficiently obtained even when the heating reaction is performed for long time.

As for the invention according to aspect 2, the height of the protruding portion is 0.1 μm to 10 μm. Consequently, the amount of the reagents and the like to be lost by entering the gaps is small and the amount can be minimized.

As for the invention according to aspect 3, the film is formed integrally with the base plate by the curable adhesive, and as for the invention according to aspect 4, the film is formed integrally with the base plate by heat-sealing. Consequently, the reaction in the reaction chamber is not disturbed and the reaction can be accurately performed.

As for the invention according to aspect 5, both the ends of the tunnel-shaped reaction chamber have the through holes penetrating the base plate. Consequently, the reagents and the like are injected into the tunnel-shaped reaction chamber from the through holes and the reaction product can be removed.

As for the invention according to aspect 6, the tunnel-shape reaction chamber serves as an amplification reaction chamber for a gene such as DNA. Consequently, the amplified gene can be sufficiently obtained as the reaction product even when the heat cycle is repeated for long time.

As for the invention according to aspects 7 to 10, the sample including a gene and the nonvolatile liquid having a light specific gravity are injected together with the gene amplification reagents to perform the amplification reaction of the gene. Consequently, even when both the ends of the tunnel-shaped reaction chamber have the through holes, the evaporation of the reaction liquid from the through holes is prevented and a required amount of the amplified gene can be obtained.

1. A reaction container comprising:
a base plate; and
a film formed integrally with the base plate,
wherein the base plate has a linear recess on a reverse side surface thereof and
the film covers the linear recess to configure a tunnel-shaped reaction chamber surrounded by the film and the base plate, and
wherein the film protrudes into the reaction chamber from the reverse side surface of the base plate to form a protruding portion.

2. The reaction container according to claim 1, wherein a height of the protruding portion is 0.1 μm to 10 μm.

3. The reaction container according to claim 1 or 2, wherein the film is formed integrally with the base plate by a curable adhesive.

4. The reaction container according to claim 1 or 2, wherein the film is formed integrally with the base plate by heat-sealing.

5. The reaction container according to claim 1 or 2, wherein both ends of the tunnel-shaped reaction chamber have through holes penetrating the base plate.

6. The reaction container according to claim 1 or 2, wherein the tunnel-shaped reaction chamber is a gene amplification reaction chamber.

7. A gene amplification reaction method comprising:
injecting a sample including a gene, a gene amplification reagent, and a nonvolatile liquid having a light specific gravity into the reaction chamber of the reaction container according to claim 5; and
performing amplification reaction of the gene.

8. The gene amplification reaction method according to claim 7, wherein the sample is a biological sample.

9. The gene amplification reaction method according to claim 7, wherein the gene amplification reagent is a PCR reaction reagent.

10. The gene amplification reaction method according to any one of claims 7, wherein the nonvolatile liquid is mineral oil, vegetable oil, or silicon oil.

11. The reaction container according to claim 3, wherein both ends of the tunnel-shaped reaction chamber have through holes penetrating the base plate.

12. The reaction container according to claim 4, wherein both ends of the tunnel-shaped reaction chamber have through holes penetrating the base plate.

13. The reaction container according to claim 3, wherein the tunnel-shaped reaction chamber is a gene amplification reaction chamber.

14. The reaction container according to claim 4, wherein the tunnel-shaped reaction chamber is a gene amplification reaction chamber.

15. The reaction container according to claim 5, wherein the tunnel-shaped reaction chamber is a gene amplification reaction chamber.

16. The gene amplification reaction method according to claim 8, wherein the gene amplification reagent is a PCR reaction reagent.

17. The gene amplification reaction method according to claim 8, wherein the nonvolatile liquid is mineral oil, vegetable oil, or silicon oil.

18. The gene amplification reaction method according to claim 9, wherein the nonvolatile liquid is mineral oil, vegetable oil, or silicon oil.
